(19) United States
(12) Patent Application Publication
(10) Pub. No.: US 2022/0183900 A1
(43) Pub. Date: Jun. 16, 2022

(54) APPARATUS AND METHOD FOR APPLYING COMPRESSION

(71) Applicant: Ilan Ben-Yaacov, Goleta, CA (US)

(72) Inventor: Ilan Ben-Yaacov, Goleta, CA (US)

(21) Appl. No.: 17/689,126

(22) Filed: Mar. 8, 2022

Related U.S. Application Data
(63) Continuation of application No. PCT/US2020/048663, filed on Aug. 30, 2020.
(60) Provisional application No. 62/901,241, filed on Sep. 16, 2019.

(51) Int. Cl. A61F 13/08; A61H 7/00; G16H 40/63

(52) U.S. Cl. CPC A61F 13/08; A61H 7/001; G16H 40/63; A61H 2209/00; A61H 2201/149; A61H 2201/5071; A61H 2205/12; A61H 2230/255; A61H 2230/655; A61H 2201/1238; A61H 2201/165; A61H 2205/106; A61H 2201/5082

(57) ABSTRACT

A compression device for applying pressure to a body part can include a fabric member that can fit over the body part, and one or more actuators coupled to the fabric member, wherein each of the one or more actuators is configured to cause at least a portion of the fabric member to expand or contract along a circumferential direction, thereby decreasing or increasing the pressure applied to the body part adjacent to the actuator. The fabric member can be thin and stretchable, and can contour the body part over which it is worn. The actuators can cause user controlled variable pressure levels to be applied to the body part, including constant pressure levels as well as time varying synchronous or asynchronous pressure patterns.

### CROSS REFERENCE TO RELATED APPLICATIONS

This application is a continuation of PCT/US2020/048663, filed Aug. 30, 2020, which claims the benefit of priority to U.S. Provisional Patent Application Ser. No. 62/901,241, filed Sep. 16, 2019, both of which are hereby incorporated by reference in their entirety.

### TECHNICAL FIELD

The current disclosure relates to devices that apply compression to a body part, and methods of using the same.

### BACKGROUND

The human body requires sufficient circulation of blood in order to maintain proper health. Improved circulation can lead to a number of health benefits, including improved organ function and removal of waste created by various organs, improved immune system functionality, increased energy, and quicker recovery after strenuous activity or after injury. Poor blood circulation, particularly in joints such as ankles and wrists, as well as nearby limbs and extremities, can lead to pain, discomfort, and reduced mobility, and in some cases can result in memory glitches, alopecia, deep vein thrombosis (DVT), or various other afflictions. Factors that can further contribute to poor circulation and the associated afflictions described above include, for example, prolonged bed rest, injury or surgery, pregnancy, obesity, age, and sitting for long periods of time, such as when driving or flying.

Compression socks (also known as compression stockings) are commonly worn in order to boost circulation in the legs, support veins, prevent blood from pooling in leg veins, prevent venous ulcers, prevent the development of DVT in the legs, or to reduce or prevent swelling in the lower legs, ankles, or feet. Compression socks come in a variety of pressure gradient levels. Mild to medium level compression socks typically provide pressure in a range of about 10-20 mmHg, firm socks provide 20-30 mmHg of pressure, extra firm socks provide 30-40 mmHg of pressure, and medical grade compression socks can provide 40-50 mmHg of pressure. While compression socks can be effective at preventing or treating many of the afflictions described above, they can be uncomfortable and can be difficult to put on and remove, particularly in the case of compression socks that provide higher levels of pressure.

### SUMMARY

Described herein are compression devices that can be worn over one or more body parts and apply variable levels of pressure to the body part, and methods of operation thereof. The devices each include a fabric member that can fit conformally over the body part and one or more actuators that control the level of pressure/compression at one or more positions in the body part beneath the compression device. The compression devices can apply constant or time varying pressure patterns to the body part in order to improve circulation, which can in some cases lead to reduced swelling and inflammation.

Accordingly, in a first aspect, a compression device configured to apply pressure to a body part can include a fabric member comprising an inner surface and an outer surface opposite the inner surface, and one or more actuators coupled to the fabric member. The fabric member may be configured to fit conformally over the body part and to be stretched while the compression device is over the body part. The one or more actuators can be configured to adjust the pressure applied to the body part.

In a second aspect, a compression device configured to apply pressure to a body part can include a fabric member configured to fit over the body part, and one or more actuators coupled to the fabric member. Each of the one or more actuators can be configured to cause at least a portion of the fabric member to expand or contract along a circumferential direction, thereby decreasing or increasing the pressure applied to the body part adjacent to the actuator.

In a third aspect, a method of operating a compression device that is configured to apply pressure to a body part is described. The compression device can include a fabric member configured to fit conformally over the body part and one or more actuators coupled to the fabric member, wherein the one or more actuators are configured to adjust the pressure applied to the body part. The method can include placing the compression device over the body part, thereby causing the fabric member to be stretched, and changing the state of at least one of the one or more actuators to a more contracted state, thereby increasing the pressure applied by the compression device to the body part.

In a fourth aspect, a method of operating a compression device that is configured to apply pressure to a body part is described. The compression device can include a fabric member configured to fit over the body part, and one or more actuators coupled to the fabric member. Each of the one or more actuators can be configured to cause at least a portion of the fabric member to expand or contract along a circumferential direction, thereby decreasing or increasing the pressure applied to the body part. The method can include causing at least one of the one or more actuators to be in an extended state prior to placing the compression device over the body part, thereby causing a portion of the fabric member adjacent to the at least one or more actuators to be expanded along the circumferential direction. The method can further include placing the compression device over the body part, and then changing the state of the at least one of the one or more actuators to a more contracted state, thereby increasing the pressure applied by the compression device to the body part.

In a fifth aspect, a method of operating a compression device that is configured to apply pressure to a body part is described. The compression device can include a fabric member configured to fit over the body part, and a plurality of actuators coupled to the fabric member, wherein each of the actuators is configured to cause at least a portion of the fabric member to expand or contract along a circumferential direction, thereby decreasing or increasing the pressure applied to the body part adjacent to the actuator. The method can include placing the compression device over the body part, and causing at least two of the actuators to apply asynchronous time varying pressure patterns to the body part. The causing of the at least two of the actuators to apply asynchronous time varying pressure patterns to the body part can be carried out by an electronic controller that is electrically coupled to the actuators.

Any of the methods described herein can include one or more of the following steps or features, either alone or in combination with one another. The method can further include adjusting the state of at least one of the one or more actuators to vary the level of pressure provided to the body part. The method can further include changing the state of the at least one of the one or more actuators to a less contracted state, and removing the compression device from the body part. The method can further include collecting, by the electronic controller, pressure, temperature, blood flow rate, or skin resistance data output by one or more sensors of the compression device. The method can further include varying, by the electronic controller, the pressure patterns applied by the actuators in response to the sensor data collected by the electronic controller during operation of the compression device.

Any of the devices and methods described herein can each include one or more of the following features, either alone or in combination with one another. The compression device can be configured such that the fabric member is stretched to an area that is at least 1.03 times (e.g., at least 1.05 times, at least 1.1 times, at least 1.15 times, at least 1.2 times, at least 1.25 times, at least 1.3 times, at least 1.35 times, at least 1.4 times, at least 1.45 times, or at least 1.5 times) its equilibrium (unstretched) area while the compression device is over the body part. The fabric member can be configured to fit conformally over the body part. The fabric member can include a first fabric portion and a second fabric portion. The first fabric portion can have a different elasticity from the second fabric portion. The first fabric portion can have a greater elasticity than the second fabric portion. The compression device can be configured such that the first fabric portion is stretched to an area that is at least 1.1 times its equilibrium area and the second fabric portion is stretched to an area that is less than 1.1 times its equilibrium area while the compression device is over the body part. The fabric member can be continuous along the circumferential direction.

At least one of the one or more actuators can be coupled (e.g., coupled directly) to the first fabric portion but not to the second fabric portion. At least one of the one or more actuators can be coupled (e.g., coupled directly) to the second fabric portion but not to the first fabric portion. At least one of the one or more actuators can be connected to an interface between the first and second fabric portions. Any of the actuators herein can include or be formed of a linear actuator, a rotary device (e.g., a motor), a pneumatic actuator, a hydraulic actuator, or a fluidic based actuator. Any of the actuators herein can further include one or more strings or wires (e.g., flexible wires). Any of the compression devices herein can be configured such that at least a portion of the fabric member is expanded in a circumferential direction while the one or more actuators are in an extended state. While the one or more actuators are in the extended state, the area of the fabric member can be at least 1.02 times the fabric member's equilibrium (unstretched) area.

The device can further include a controller coupled to each of the one or more actuators. The controller can be an electronic controller that is electrically coupled to each of the one or more actuators. The controller can be directly coupled to, or can be on, over, or embedded within the fabric member. The controller can be configured to cause the one or more actuators to vary the pressure applied to the body part while the compression device is worn by a user. The one or more actuators can include at least two actuators, and the controller can cause each of the at least two actuators to apply time varying pressure patterns. The time varying pressure patterns applied by each of the at least two actuators can be asynchronous. The device can further include one or more sensors that measure pressure, temperature, or skin resistance. Pressure, temperature, or skin resistance data output by the one or more sensors can be collected or stored by the electronic controller. The electronic controller can be configured to vary the pressure patterns applied by the actuators in response to the sensor data collected or stored by the electronic controller during operation of the compression device. The device can further include a battery. The battery can be directly coupled to, or can be on, over, or embedded within the fabric member. The battery can be removable from the fabric member. The battery can be configured to be recharged via the electronic controller. The causing of the at least two of the actuators to apply asynchronous time varying pressure patterns to the body part can be carried out by an electronic controller that is electrically coupled to the actuators. The method can further include collecting or storing, by the electronic controller, pressure, temperature, or skin resistance data output by one or more sensors of the compression device. The method can further include varying, by the electronic controller, the pressure patterns applied by the actuators in response to the sensor data collected or stored by the electronic controller during operation of the compression device.

The pressure provided by the compression device can vary over the length of the compression device. For example, the pressure provided by the compression device can vary monotonically from a first end of the compression device to a second end of the compression device. Each of the one or more actuators can be configured to cause at least a portion of the fabric member to expand or contract along a circumferential direction, thereby decreasing or increasing the pressure applied to the body part adjacent to the actuator. A first end of the fabric member can include an opening through which the body part is inserted, and a second end of the fabric member which is opposite the first end can be closed. At least 95% of an inner surface of the fabric member can directly contact the body part while the compression device is worn over the body part. A separation between the outer surface and the inner surface of the fabric member can be less than 3.5 centimeters (cm), e.g., less than 3 cm, less than 2.5 cm, less than 2 cm, less than 1.5 cm, less than 1 cm, less than 8 millimeters (mm), less than 6 mm, or less than 5 mm while the compression device is over the body part with the inner surface contacting the body part.

### DESCRIPTION OF DRAWINGS

FIGS. 1 and 2 are illustrations of exemplary compression devices.

FIGS. 3-8 are diagrams of exemplary actuator configurations that can be used with the compression devices of FIGS. 1 and 2.

FIG. 10 is an illustration of an optional configuration for a portion of the device of FIG. 2.

FIGS. 9, 11 and 12 are flow charts illustrating methods of operating a compression device.

Like numbers in the drawings represent like elements.

### DETAILED DESCRIPTION

Described herein are compression devices that can be worn over one or more body parts and apply variable levels of pressure to the body part, and methods of operation thereof. The devices each include a fabric member that can fit conformally over the body part and one or more actuators that control the level of pressure/compression at one or more positions in the body part beneath the compression device. In some cases, the level of pressure/compression applied to the body part at various positions beneath the device is actively controlled and/or varied while the device is worn. By applying specific time varying pressure patterns, circulation in the body part can be improved, and in some cases swelling and inflammation can be reduced.

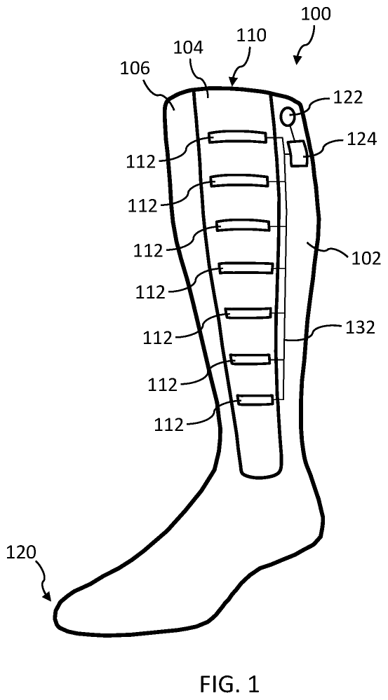

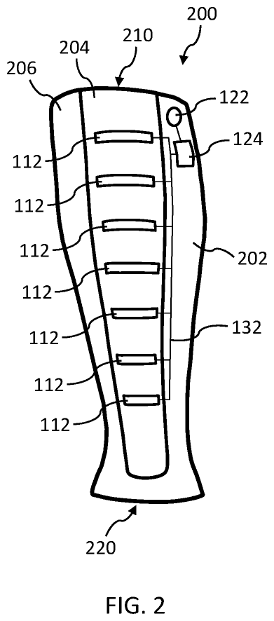

An exemplary compression device 100 is illustrated in FIG. 1. Compression device 100 includes a fabric member 102 that fits over and covers the body part to which the pressure is applied by the device. The fabric member 102 includes an inner surface that contacts the body part over which it is worn and an outer surface on the opposite side of the fabric member from the inner surface, with the outer surface typically exposed to air while the device is worn. In the example shown in FIG. 1, the fabric member 102 is in the shape of a sock that slides over a foot and covers at least a portion of the leg. That is, a first end 110 of the fabric member can include an opening through which the foot and leg are inserted, a second end 120 of the fabric member which is opposite the first end is closed, and the fabric member wraps continuously around the foot and lower leg without requiring an attachment mechanism (e.g., hook and loop fastener) to keep the device in place. However, other configurations and shapes are possible as well. For example, FIG. 2 shows another compression device 200 which is similar to that of FIG. 1, except that the fabric member 202 of device 200 has openings at both ends 210 and 220, such that the foot passes through both openings when putting the device on, and the device acts as a sleeve covering the lower (and optionally the upper) portion of the leg without covering the foot. Additionally, the specific shape of the fabric members 102 and 202 of either of devices 100 and 200 could be modified to allow the compression device to fit comfortably over other body parts, for example hands, fingers, and/or arms. That is, the fabric members 102 and 202 of either of compression devices 100 and 200 could be modified to be shaped, e.g., as a glove or as a sleeve that covers at least a portion of a hand and/or an arm.

The fabric member 102/202 can be configured to fit conformally over the body part that is covered by the compression device. That is, the fabric member can be sufficiently elastic to allow it to stretch and take the form of the surface of the body part that is covered while applying pressure/compression to the body part. For any of the compression devices described herein, at least 95% (e.g., at least 96%, at least 97%, at least 98% or at least 99%) of the inner surface of the fabric member can contact (e.g., directly contact) the body part while the compression device is worn and applies pressure/compression to the body part.

The fabric member 102/202 can be made thin, and can, e.g., be of similar thickness to a conventional sock, thereby allowing the compression device 100/200 to be worn underneath clothing and otherwise have a low profile. For example, the thickness of the fabric member (e.g., the separation between the outer surface and inner surface of the fabric member) can everywhere be less than 3.5 centimeters (cm), less than 3 cm, less than 2.5 cm, less than 2 cm, less than 1.6 cm, less than 1.2 cm, 1 cm, less than 8 millimeters (mm), less than 6 mm, less than 4 mm, less than 2 mm, or less than 1 mm.

As seen in FIGS. 1 and 2, the fabric member 102/202 of the compression device 100/200 can include or be formed of a first fabric portion 104/204 and a second fabric portion 106/206. The first and second fabric portions 104/204 and 106/206 can have a different elasticity (i.e., “stretchiness”) from one another. For example, the first fabric portion 104/204 can have a greater elasticity (i.e., can be more “stretchy”) than the second fabric portion 106/206. As further described below, this can allow for controlled levels of pressure to be maintained on the body part at various positions in the compression device without excessive stretching of the fabric member 102/202.

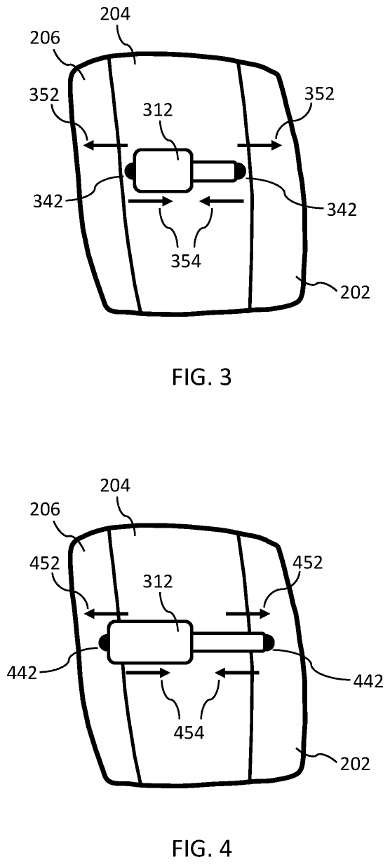

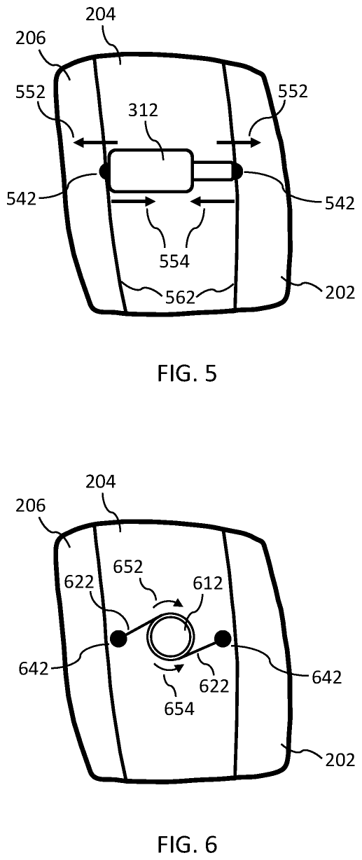

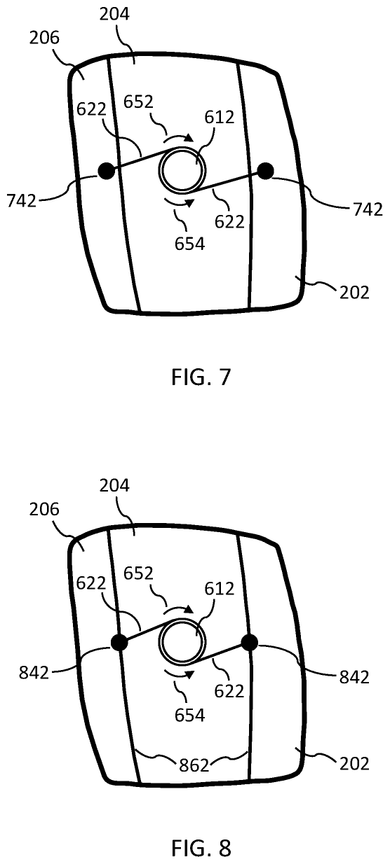

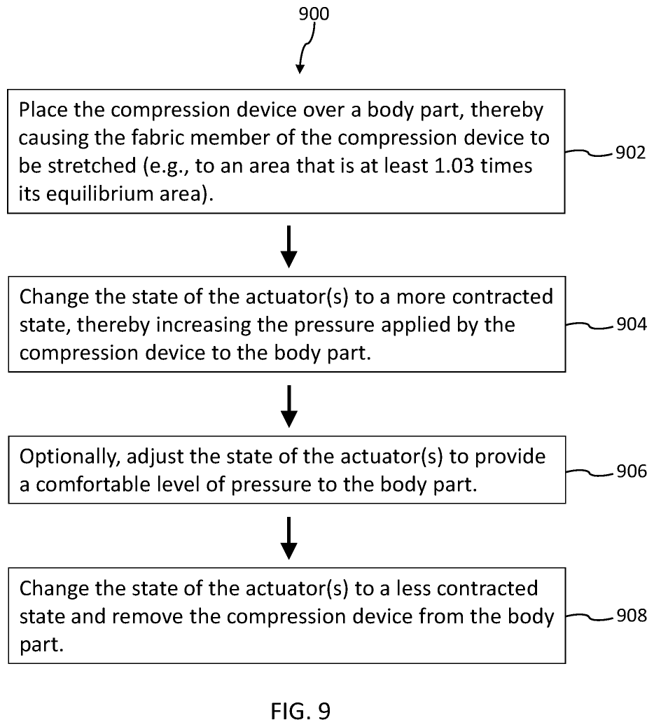

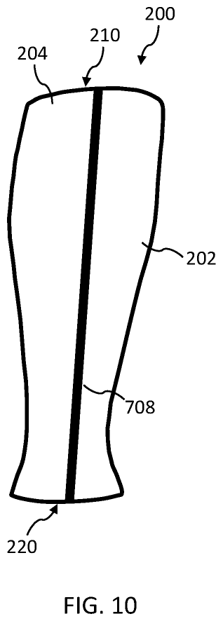

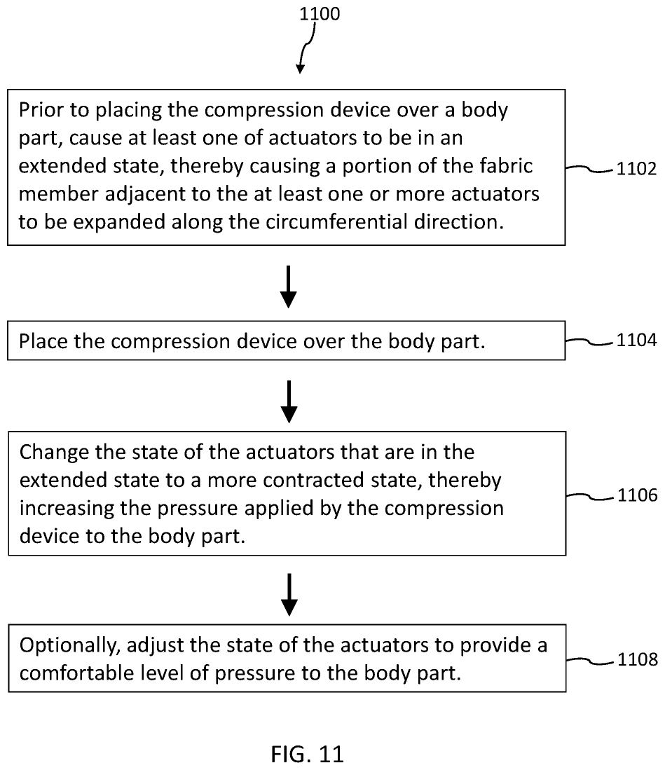

As further seen in FIGS. 1 and 2, compression devices 100 and 200 include one or more actuators 112. While FIGS. 1 and 2 represent the actuators as elongated strips, the exact size and shape of the actuators will depend on the specific type of actuator used, as further described below. The actuators 112 can be any device that can adjust the state of the compression device 100 or 200 to apply more or less pressure to the body part over which the compression device is placed. Each of the actuators 112 causes the compression device 100/200 to expand and/or contract in a circumferential direction (i.e., around the circumference of the body part) directly below or adjacent to the actuator, thereby varying (e.g., increasing or decreasing) the pressure applied adjacent to the actuator. As described below and shown in FIGS. 3 and 6, the actuators 112 can be coupled to (e.g., connected to) the first fabric portion 104/204 but not to the second fabric portion 106/206, and optionally the first fabric portion 104/204 can have a greater elasticity than the second fabric portion 106/206. Alternatively, as also described below and shown in FIGS. 4 and 7, the actuators 112 can be coupled to (e.g., connected to) the second fabric portion 106/206 but not to the first fabric portion 104/204, and optionally the first fabric portion 104/204 can have a greater elasticity than the second fabric portion 106/206. Or, as described below and shown in FIGS. 5 and 8, the actuators can be coupled to (e.g., connected to) the interface between the first fabric portion 104/204 and the second fabric portion 106/206. These configurations can allow the first fabric portion 104/204 to stretch or contract in a circumferential direction, while the second fabric portion 106/206 remains more rigid, thereby requiring less stretching of the fabric member 102/202 by the actuators 112 in order to provide the requisite pressure/compression to the underlying body part.

The actuators 112 can each, for example, include or be formed as a linear actuator, a rotary device, or a motor (e.g., a linear or rotary motor). FIGS. 3-8 are diagrams of exemplary actuator configurations that can be used with the compression devices of FIGS. 1 and 2. FIG. 3 shows a linear actuator 312 coupled to (e.g., connected to) fabric member 202, and specifically to the first fabric portion 204 but not to the second fabric portion 206. The linear actuator 312 is connected to the first fabric portion 204 of fabric member 202 at points 342. In FIG. 3, the fabric member 202 is shown in its equilibrium position when the device is worn by a user. That is, the linear actuator 312 is extended to a point where it applies no circumferential force to the fabric member 202, and all pressure applied by the compression device 200 to the body part results entirely from the stretch of the fabric member 202. Further extending the linear actuator 312 in the direction indicated by arrows 352, such that the actuator 312 is in an extended state, stretches the first fabric portion 204 (i.e., causes the first fabric portion 204 to expand), causing the pressure applied by the compression device to the body part adjacent to the actuator to decrease. If the second fabric portion 206 is formed of an elastic material, actuation of the linear actuator 312 in this direction can also cause some contraction of the second fabric portion 206. While the actuator 312 is in the extended state, the fabric member 202 may be stretched to have an area that is at least 1.02 times (e.g., at least 1.03 times, at least 1.05 times, at least 1.1 times, at least 1.15 times, at least 1.2 times, at least 1.25 times, at least 1.3 times, at least 1.35 times, at least 1.4 times, at least 1.45 times, or at least 1.5 times) its equilibrium (unstretched) area. Compressing the linear actuator 312 in the direction indicated by arrows 354, such that the actuator 312 is in a contracted state, causes the second fabric portion 206 to be under increased tensile stress, thereby causing the pressure applied by the compression device to the body part adjacent to the actuator to increase.

FIG. 4 shows a linear actuator 312 coupled to (e.g., connected to) the second fabric portion 206 but not to the first fabric portion 204. The linear actuator 312 is connected to the second fabric portion 206 of fabric member 202 at points 442. In FIG. 4, the fabric member 202 is shown in its equilibrium position when the device is worn by a user. That is, the linear actuator 312 is extended to a point where it applies no circumferential force to the fabric member 202, and all pressure applied by the compression device 200 to the body part results entirely from the stretch of the fabric member 202. Further extending the linear actuator 312 in the direction indicated by arrows 452 stretches the first fabric portion 204 (i.e., causes the first fabric portion 204 to expand), causing the pressure applied by the compression device to the body part adjacent to the actuator to decrease. If the second fabric portion 206 is formed of an elastic material, actuation of the linear actuator 312 in this direction can also cause some contraction of the second fabric portion 206. While the actuator 312 is in the extended state, the fabric member 202 may be stretched to have an area that is at least 1.03 times (e.g., at least 1.05 times, at least 1.1 times, at least 1.15 times, at least 1.2 times, at least 1.25 times, at least 1.3 times, at least 1.35 times, at least 1.4 times, at least 1.45 times, or at least 1.5 times) its equilibrium (unstretched) area. Compressing the linear actuator in the direction indicated by arrows 454 causes the second fabric portion 206 to be under increased tensile stress, thereby causing the pressure applied by the compression device to the body part adjacent to the actuator to increase.

FIG. 5 shows a linear actuator 312 coupled to (e.g., connected to) the interface 562 between the first fabric portion 204 and the second fabric portion 206. The linear actuator 312 is connected to interface 562 at points 542. In FIG. 5, the fabric member 202 is shown in its equilibrium position when the device is worn by a user. That is, the linear actuator 312 is extended to a point where it applies no circumferential force to the fabric member 202, and all pressure applied by the compression device 200 to the body part results entirely from the stretch of the fabric member 202. Further extending the linear actuator 312 in the direction indicated by arrows 552 stretches the first fabric portion 204 (i.e., causes the first fabric portion 204 to expand), causing the pressure applied by the compression device to the body part adjacent to the actuator to decrease. If the second fabric portion 206 is formed of an elastic material, actuation of the linear actuator 312 in this direction can also cause some contraction of the second fabric portion 206. While the actuator 312 is in the extended state, the fabric member 202 may be stretched to have an area that is at least 1.03 times (e.g., at least 1.05 times, at least 1.1 times, at least 1.15 times, at least 1.2 times, at least 1.25 times, at least 1.3 times, at least 1.35 times, at least 1.4 times, at least 1.45 times, or at least 1.5 times) its equilibrium (unstretched) area. Compressing the linear actuator in the direction indicated by arrows 554 causes the second fabric portion 206 to be under increased tensile stress, thereby causing the pressure applied by the compression device to the body part adjacent to the actuator to increase.

FIGS. 6-8 illustrate the use of a rotary device 612 (e.g., a motor) and one or more strings/wires 622 as an actuator. The strings/wires 622 may be flexible. In FIG. 6, the strings/wires 622 are connected to the rotary device 612, and are also coupled (e.g., connected) to the first fabric portion 204 of fabric member 202 at points 642. In FIG. 7, the strings/wires 622 are connected to the rotary device 612, and are also coupled (e.g., connected) to the second fabric portion 206 of fabric member 202 at points 742. In FIG. 8, the strings/wires 622 are connected to the rotary device 612, and are also coupled (e.g., connected) to the interface 862 between the first fabric portion 204 and the second fabric portion 206 at points 842. Rotating the rotary device 612 in the direction indicated by arrow 652 causes the actuator to be in a more contracted state, thereby increasing the pressure applied by the compression device to the body part adjacent to the actuator, while rotating the motor in the direction indicated by arrow 654 causes the actuator to be in a more extended state, thereby decreasing the pressure applied by the compression device to the body part adjacent to the actuator.

Apart from the actuators shown in FIGS. 3-8, other types of actuators could additionally or alternatively be used. For example, pistons or other pneumatic or hydraulic or fluidic based actuators could be used to achieve the desired variable pressure levels provided by the compression device.

Referring back to FIGS. 1 and 2, compression devices 100 and 200 can be placed over the body part (e.g., the arm or leg) by sliding the device on. For example, for a compression device configured to fit over at least a portion of the leg, the user can slide the device over the foot and onto the leg, and then position the device where desired. For a compression device configured to fit over at least a portion of the arm, the user can slide the device over the hand and onto the arm, and then position the device where desired. As such, the fabric member 202 can extend around the body part continuously in a circumferential direction, without a slit or opening that requires an attachment mechanism to keep the device over the body part.

The fabric member 102/202 of compression device 100/200 can be configured to apply some pressure to the body part over which the compression device 100/200 is placed prior to any of the actuators 112 being engaged, and one or more of the actuators 112 can then be engaged in order to increase the pressure applied by the compression device 100/200. For example, when compression device 100/200 is placed over the body part with the actuators in an extended state, the device 100/200 can fit conformally over the body part with the fabric member 102/202 stretched to have an area that is at least 1.03 times (e.g., at least 1.05 times, at least 1.1 times, at least 1.15 times, at least 1.2 times, at least 1.25 times, at least 1.3 times, at least 1.35 times, at least 1.4 times, at least 1.45 times, or at least 1.5 times) its equilibrium (unstretched) area. Once the device 100/200 is over the body part, one or more of the actuators 112 can be engaged in order to increase the pressure applied to the body part. This can allow the compression device 100/200 to be easily put on and taken off while still applying a substantial amount of pressure while the device is in use, and can also allow the pressure applied by the device to be readily adjusted to a comfortable level by the user.

The stretching of the fabric member 102/202 that occurs while the compression device 100/200 is initially placed over the body part can primarily occur in the first fabric portion 104/204. That is, when the device is initially placed over the body part, the percent stretch of the first fabric portion 104/204 can be greater than the percent stretch of the second fabric portion 106/206. For example, the first fabric portion 104/204 can be stretched to an area that is at least 1.05 times its equilibrium (unstretched) area while the second fabric portion 106/206 is not substantially stretched or is stretched to an area that is less than 1.05 times its equilibrium (unstretched) area. Or, the first fabric portion 104/204 can be stretched to an area that is at least 1.1 times its equilibrium (unstretched) area while the second fabric portion 106/206 is not substantially stretched or is stretched to an area that is less than 1.1 times its equilibrium (unstretched) area. Or, the first fabric portion 104/204 can be stretched to an area that is at least 1.2 times its equilibrium (unstretched) area while the second fabric portion 106/206 is not substantially stretched or is stretched to an area that is less than 1.2 times its equilibrium (unstretched) area. Or, the first fabric portion 104/204 can be stretched to an area that is at least 1.3 times its equilibrium (unstretched) area while the second fabric portion 106/206 is not substantially stretched or is stretched to an area that is less than 1.3 times its equilibrium (unstretched) area.

In view of the above, FIG. 9 is a flow diagram of a method 900 of operating a compression device (e.g., device 100 or 200 of FIG. 1 or FIG. 2). First, the compression device is placed over a body part, thereby causing the fabric member (e.g., fabric member 102 or 202) of the compression device to be stretched, e.g., to an area that is at least 1.03, 1.05, or 1.1 times its equilibrium area (step 902). Next, the state of some or all of the actuators 112 of the compression device 100/200 are changed to a more contracted state, thereby increasing the pressure applied by the compression device to the body part (step 904). The state of each actuator can optionally be adjusted to provide a comfortable level of pressure to the body part (step 906) while the device is worn. Finally, when the user wishes to remove the compression device, the state of each actuator 112 is returned to a less contracted state, and the compression device is removed from the body part (step 908).

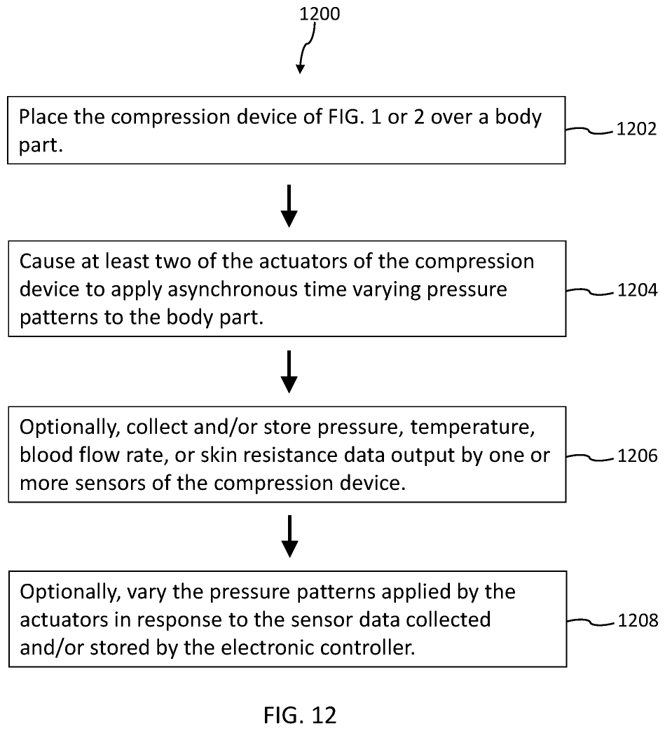

The fabric member 202 of the compression device 200 may optionally include a slit or opening with an attachment mechanism that allows the device to be attached over the body part without sliding the device over the body part. For example, FIG. 10 illustrates an optional configuration for the portion of device 200 that is on the opposite side of the body part from the portion illustrated in FIG. 2. As seen in FIG. 10, the fabric member 202 can include a slit/opening 708 which includes an attachment mechanism such as hook and loop fastener (e.g., Velcro), a zipper, one or more buckles, or the like. The user can thereby place the device 200 over the body part by wrapping the device around the body part and connecting the portions of the fabric member 202 on opposite sides of the slit/opening 708 via the attachment mechanism. In this configuration, the fabric member 202 does not continuously extend circumferentially around the body part over which the compression device is worn, as it includes slit/opening 708 and thus requires an attachment mechanism to hold the device in place around the body part.

The configuration shown in FIG. 10 can offer several benefits. For example, it can be easier to strap the device over the arm or leg than to pull it over the hand or foot. Furthermore, the configuration of FIG. 10 can allow users to attach the device to their leg without having to remove shoes that they might be wearing. Additionally, the device can be readily strapped on over another sock or sleeve (e.g., a compression sock or a compression sleeve), thereby allowing the user to wear a passive device underneath the device 200 of FIGS. 2 and 10.

In implementations where the compression device 100/200 only includes a single actuator, the actuator design or the material design used for the fabric member 202 can be configured such that the increased pressure provided by the actuator is provided over a substantial percentage of the area of the fabric member 202 (e.g., over at least 30%, at least 40%, at least 50%, at least 60%, at least 70%, at least 80%, or at least 90% of the area of the fabric member 102/202). In some implementations where the compression device 100/200 includes multiple actuators, the actuators can be coupled to one another such that they all provide the same state of compression or expansion at the same time, which can allow a single control signal to simultaneously control all of the actuators. For each of these implementations, the compression device 100/200 can be operated as a passive pressure variable device, thereby allowing ease of use in putting on the device and positioning the device over the body part and then allowing the user to increase or adjust the level of pressure applied by the device to the body part via the actuator(s).

In view of the above, FIG. 11 is a flow diagram of a method 1100 of operating a compression device (e.g., device 100 or 200 of FIG. 1 or FIG. 2). Prior to placing the compression device over the body part, at least one of the actuators is caused to be in an extended state (step 1102), corresponding to the state in which the circumference of the compression device is expanded over at least a portion of the length of the device (i.e., causing a portion of the fabric member adjacent to the actuator(s) to be expanded along the circumferential direction). This can allow the compression device to be more easily put on, as it can be loose fitting. Next, the compression device is placed over the body part (step 1104), for example by sliding the device over the body part while it is in its loose-fitting state. Once the compression device is positioned over the body part, the actuators that are in the extended state are changed to a more contracted state (step 1106), corresponding to a state in which the circumference of the compression device over at least a portion of the length of the device is less expanded or is contracted. This increases the pressure applied by the compression device to the body part. The state of the actuators can optionally be adjusted so that the compression device provides a comfortable level of pressure/compression to the body part (step 1108).

The actuator(s) can be configured to provide varying pressure over the length of the compression device. That is, any of the compression devices described herein can be configured such that after the device is placed over the body part and the actuator(s) are actuated to provide pressure to the body part, different levels of pressure are provided to different portions of the body part, even if the pressure is not further varied over time. For example, the compression device can be configured to provide a gradient pressure over the length of the device. In some implementations where the compression device is worn over the lower leg (e.g., device 100 or 200 of FIGS. 1 and 2), the compression device provides a first pressure on the end 120/220 proximal to the foot, and the device provides a second pressure that is less than the first pressure on the end 110/210 that is higher up the leg. Optionally the provided pressure decreases monotonically from the first pressure to the second pressure over the length of the compression device.

When the compression device is configured to be worn over other body parts (e.g., an arm), the compression device can provide a static pressure that varies over the length of the device. The pressure can, for example, vary (i.e., increase or decrease) monotonically from one end (e.g., the first end) of the device to the other end (e.g., the second end) of the device. Static pressure with pressure levels that vary over the length of the device can allow for optimal comfort and support while the device is worn, as well as improving blood flow in the body part over which the device is worn.

While each of the actuators 112 of compression device 100/200 can be controlled individually, either manually (e.g., mechanically) or electronically, the compression device 100/200 can optionally be configured to automatically apply varying compression levels at or near the various actuators. For example, the compression device 100/200 can include a controller 124 that is coupled to and controls some or all of the actuators 112. The controller 124 can, for example, be an electronic controller that is electrically coupled to one or more of the actuators 112. The controller 124 can, for example, be a microcontroller that is connected to the actuators 112 via electrical connectors 132, thereby directing power and/or control signals to the actuators 112. The controller can be directly coupled to the fabric member 102/202, or can be embedded within or placed on or over the fabric member 102/202. The controller 124 and actuators 112 can be powered by a battery 122. The battery can also be directly coupled to the fabric member 102/202, or can be embedded within or placed on or over the fabric member 102/202. As such, in addition to being thin and having a low profile, the compression device 100/200 can be highly versatile and portable, as all elements of the device can be directly on or adjacent to or embedded within the fabric member 102/202. Additionally, the battery can be removable and/or rechargeable. For example, the controller 124 can include a charging port through which the battery 122 can be charged.

As described above, the controller 124 can be configured to cause the one or more actuators 112 to vary the pressure applied to the body part while the compression device 100/200 is worn by a user. For example, the controller 124 can be configured to cause all of the actuators to simultaneously and synchronously increase and then decrease pressure on the body part repeatedly, thereby creating a pulsating pressure sensation over the entire body part. This can allow for higher pressure to be applied for short periods of time than could be tolerated by a device that applies a constant pressure. For example, a pressure of at least 60 mmHg, at least 70 mmHg, at least 80 mmHg, at least 90 mmHg, or at least 100 mmHg may be applied for relatively short periods of time (e.g., less than 20 minutes, less than 18 minutes, less than 16 minutes, less than 14 minutes, less than 12 minutes, less than 10 minutes, less than 8 minutes, less than 7 minutes, less than 6 minutes, less than 5 minutes, less than 4 minutes, less than 3 minutes, less than 2 minutes, or less than 1 minute), followed by lower pressure (e.g., less than 50 mmHg, less than 40 mmHg, less than 30 mmHg, less than 20 mmHg, or less than 10 mmHg) applied for some amount of time (e.g., at least 1 minute, at least 2 minutes, at least 5 minutes, at least 10 minutes, at least 15 minutes, at least 20 minutes, or in a range of 1 to 20 minutes), with the process repeating itself as many times as desired.

The controller 124 can also or alternatively be configured to cause at least two of the actuators to apply asynchronous time varying pressure patterns. For example, when the compression device 100/200 is configured to fit over the lower leg, the controller 124 can be programmed to create a pressure wave that moves up and/or down the leg. That is, actuators can be programmed to contract consecutively from bottom to top (or from top to bottom) and then expand consecutively from bottom to top (or from top to bottom). Such a pressure wave can help increase circulation and can be particularly useful in the lower legs, which are more susceptible than other areas of the body to afflictions such as swelling, edema, and DVT. Because the compression device 100/200 can be pre-programmed to apply these pressure patterns without requiring subsequent user input, the device can be used passively while the user is able to perform other activities (e.g., working at a desk, sitting on an airplane) without needing to attend to the compression device.

When an electronic controller such as a microcontroller is used to control the state of the actuators, the controller can include a memory, e.g., for storing various actuator control sequences, and a computing device including at least one processor, e.g., a microprocessor, for sending/applying the stored control signals to the actuators.

Although not pictured in FIG. 1, 2, or 10, the compression device 100/200 can optionally include sensors such as pressure sensors that detect the pressure being applied by the device to the body part at one or more positions/locations. The compression device 100/200 can also optionally include temperature sensors that measure the skin temperature of the body part beneath the compression device at one or more positions/locations. The compression device 100/200 can also optionally include sensors that measure the skin resistance of the body part beneath the compression device at one or more positions/locations. The compression device 100/200 can also optionally include sensors that measure the absolute or relative rate of blood flow beneath the compression device at one or more positions/locations. The pressure, temperature, blood flow rate, and/or skin resistance data output by the sensors can be collected and stored (e.g., in memory) by the electronic controller. The sensor data can also provide real-time feedback which can be used by the controller to adjust the pressure patterns provided by the actuators in order to maximize the performance and comfort of the compression device.

FIG. 12 illustrates another method of operating a compression device (e.g., device 100 or 200 of FIG. 1 or 2) that makes use of the asynchronous operation of the various actuators described above. First, the compression device is placed over a body part (step 1202), for example an arm or a leg (or a portion of an arm or a leg). The method then includes causing at least two of the actuators to apply asynchronous time varying pressure patterns to the body part (step 1204). As previously described, an electronic controller can be used to control the actuators and to cause them to apply the asynchronous time varying pressure patterns. The method can optionally include collecting and/or storing pressure, temperature, blood flow rate, or skin resistance data output by one or more sensors of the compression device (step 1206). The collecting and/or storing of the data can be carried out by the electronic controller. The method can also optionally include varying the pressure patterns applied by the actuators in response to the sensor data collected and/or stored by the electronic controller (step 1208). The varying of the pressure patterns can be controlled and/or carried out by the electronic controller, e.g., via electrical signals applied by the electronic controller. For example, if the sensors detect that the pressure patterns produce a maximum pressure that is greater than a desired or threshold value, the electronic controller can reduce the peak compression applied by one or more of the actuators during execution of the pressure sequence.

Any of the compression devices described herein can have one or more of the following features or advantages. The devices can be low profile, and can, for example, conform to and/or contour the body part over which they are worn, and/or be very thin. For example, the average thickness of the device (including any components such as the actuators, controller, battery, and/or sensors that are integrated into the device) while worn by a user can be less than 3 cm, less than 2.5 cm, less than 2 cm, less than 1.6 cm, less than 1.2 cm, less than 1 cm, less than 8 mm, less than 6 mm, less than 4 mm, or less than 2 mm. Accordingly, the device can be worn underneath clothing (e.g., underneath pants or shirt sleeves) without being easily visible, and can be highly versatile and portable. The compression device can optionally include a separate or removable/detachable washable inner liner over which the rest of the device can be worn, so that the user can keep the inner liner clean without needing to wash and potentially damage the rest of the device.

Various implementations of compression devices and associated methods of operation have been described above. However, it should be understood that they have been presented by way of example only, and not limitation. Where methods and steps described above indicate certain events occurring in certain order, those of ordinary skill in the art would recognize that the ordering of certain steps may be modified and such modifications are in accordance with the variations of the disclosure. The implementations have been particularly shown and described, but it will be understood that various changes in form and details may be made. Accordingly, other implementations are within the scope of the following claims.

What is claimed is:

1. A compression device configured to apply pressure to a body part, comprising:
a fabric member comprising a first fabric portion and a second fabric portion, the first fabric portion having a different elasticity from the second fabric portion, wherein the first and second fabric portions each comprise an inner surface and an outer surface opposite the inner surface; and
one or more actuators coupled to the fabric member; wherein
the fabric member is configured to fit conformally over the body part and to be stretched while the compression device is over the body part;
the one or more actuators are configured to adjust the pressure applied by the compression device to the body part; and
the compression device is configured such that while the compression device is over the body part with the fabric member stretched, actuating at least one of the one or more actuators causes the first fabric portion to expand and the second fabric portion to contract.

2. The compression device of claim 1, wherein the compression device is configured such that the fabric member is stretched to an area that is at least 1.03 times its equilibrium area while the compression device is over the body part.

3. The compression device of claim 1, wherein the compression device is configured such that the first fabric portion is stretched to an area that is at least 1.1 times its equilibrium area and the second fabric portion is stretched to an area that is less than 1.1 times its equilibrium area while the compression device is over the body part.

4. The compression device of claim 1, wherein the fabric member is continuous along a circumferential direction.

5. The compression device of claim 1, wherein at least one of the one or more actuators are selected from the group consisting of a pneumatic actuator, a hydraulic actuator, a fluidic based actuator, a linear actuator, and a rotary device.

6. The compression device of claim 1, wherein at least one of the one or more actuators comprises one or more flexible strings or wires.

7. The compression device of claim 1, wherein the one or more actuators is connected to an interface between the first and second fabric portions.

8. The compression device of claim 1, further comprising an electronic controller that is electrically coupled to each of the one or more actuators.

9. The compression device of claim 1, further comprising one or more sensors that measure pressure, temperature, blood flow rate, or skin resistance.

10. The compression device of claim 1, wherein the pressure provided by the compression device increases monotonically from a first end of the compression device to a second end of the compression device.

11. The compression device of claim 1, wherein a first end of the fabric member comprises an opening through which the body part is inserted, and a second end of the fabric member which is opposite the first end is closed.

12. The compression device of claim 1, wherein a separation between the outer surface and the inner surface of each of the first and second fabric portions is less than 1 centimeter while the compression device is over the body part with the inner surfaces of the first and second fabric portions contacting the body part.

13. A method of operating a compression device that is configured to apply pressure to a body part, the compression device comprising:
a fabric member comprising a first fabric portion and a second fabric portion, the first fabric portion having a different elasticity from the second fabric portion, wherein the first and second fabric portions each comprise an inner surface and an outer surface opposite the inner surface; and
one or more actuators coupled to the fabric member; the method comprising:
placing the compression device over the body part, thereby causing the fabric member to fit conformally over the body part and to be stretched; and
actuating at least one of the one or more actuators, thereby adjusting the pressure applied by the compression device to the body part; wherein
the actuating of the at least one of the one or more actuators causes the first fabric portion to expand and the second fabric portion to contract.

14. The method of claim 13, wherein the one or more actuators is connected to an interface between the first and second fabric portions.

15. The method of claim 13, further comprising adjusting the state of at least one of the one or more actuators to vary the level of pressure provided to the body part.

16. The method of claim 13, wherein the pressure applied by the compression device to the body part increases monotonically from a first end of the compression device to a second end of the compression device.

17. The method of claim 13, wherein at least one of the one or more actuators comprises one or more flexible strings or wires.

18. The method of claim 13, wherein the causing of the fabric member to be stretched when the compression device is placed over the body part comprises causing an area of the fabric member to be at least 1.03 times its equilibrium area.

19. The method of claim 13, wherein the actuating of the at least one of the one or more actuators causes the first fabric portion to be stretched to an area that is at least 1.1 times its equilibrium area and the second fabric portion to be stretched to an area that is less than 1.1 times its equilibrium area.

20. The method of claim 13, wherein the fabric member is continuous along a circumferential direction.
